(19) United States
(12) Patent Application Publication
(10) Pub. No.: US 2017/0095182 A1
(43) Pub. Date: Apr. 6, 2017

(54) RH INCOMPATIBILITY DETECTION

(71) Applicant: HEWLETT-PACKARD DEVELOPMENT COMPANY, L.P., HOUSTON, TX (US)

(72) Inventor: SATWANT KAUR, MOUNTAIN VIEW, CA (US)

(21) Appl. No.: 15/308,300

(22) PCT Filed: Jun. 17, 2014
(86) PCT No.: PCT/US2014/042746

§ 371 Date: Nov. 1, 2016

(51) Int. Cl. A61B 5/1455; A61B 5/00; A61B 5/145

(52) U.S. Cl. CPC A61B 5/1455; A61B 5/14546; A61B 5/681; A61B 5/6826; A61B 5/6816; A61B 2503/02

(57) ABSTRACT

One example implementation detects Rh incompatibility between a mother and a baby by utilizing a light emitter to project light into the flesh of a user, a light detector to measure voltage from light in the flesh of the user caused by the projected light, and an Rh analyzer to detect Rh incompatibility between a user and a fetus within the user based on the voltage.

### BACKGROUND

An Rh factor is a characteristic of a human blood system known as the Rhesus (Rh) blood group system. An individual is Rh positive or Rh negative, depending on the presence of the Rh factor on the surface of red blood cells of the individual. The Rh factor refers to a D antigen of the Rh blood group. An individual that is Rh positive has the D antigen on the surface of his or her red blood cells, and an individual that is Rh negative does not have the D antigen.

### BRIEF DESCRIPTION OF THE DRAWINGS

FIG. 1 is a block diagram of an example Rh incompatibility detection system, including an example Rh analyzer, constructed in accordance with an aspect of the disclosure.

FIG. 2A illustrates an example device that is worn on a human ear and that may implement the Rh incompatibility system of FIG. 1 in accordance with an aspect of the disclosure.

FIGS. 2B-2C illustrate an example device that is worn on a human hand and that may implement the Rh incompatibility detection system of FIG. 1 in accordance with an aspect of the disclosure.

FIG. 2D illustrates an example device that is worn on a human wrist and that may implement the Rh incompatibility detection system of FIG. 1 in accordance with an aspect of the disclosure.

FIG. 3 illustrates an example device that may be used to implement the devices of FIGS. 2B-2D in accordance with an aspect of the disclosure.

FIG. 4 is a block diagram of an example Rh analyzer that may implement the Rh analyzer of FIG. 1 in accordance with an aspect of the disclosure.

FIG. 5 is a flowchart representative of example machine readable instructions that may be executed to implement the Rh analyzer of FIGS. 1 and/or 4 in accordance with an aspect of the disclosure.

FIG. 6 is a flowchart representative of example machine readable instructions that may be executed to implement a portion of the example machine readable instructions of FIG. 5 in accordance with an aspect of the disclosure.

FIG. 7 is a block diagram of a processor platform capable of executing the instructions of FIGS. 5 and/or 6 to implement the Rh analyzer of FIGS. 1 and/or 4 in accordance with an aspect of the disclosure.

### DETAILED DESCRIPTION

Examples are disclosed herein to monitor and/or detect Rh incompatibility between a pregnant woman and her in utero baby. An example system or device includes a light emitter, a light detector, and an Rh analyzer that analyzes voltages and/or voltage changes in the woman from light captured from flesh and/or a bloodstream of the woman. Based on the detected voltages and/or voltage changes, example methods, systems, and articles of manufacture are used to determine that there is an Rh incompatibility (or Rh compatibility) between the woman and her baby.

As used herein, a user may be a pregnant woman carrying a fetus, and Rh incompatibility for compatibility) is monitored/detected between the woman and the fetus. For ease of readability, a user monitored for Rh incompatibility may be referred to herein as a woman or mother, and the fetus may be referred to herein as a baby. Accordingly, the terms mother and woman may be used it and the terms fetus and baby may be used interchangeably. In some examples herein, a user may be a healthcare provider of a mother.

Rh incompatibility between a pregnant mother and her in utero baby exists when the mother has Rh negative blood and her baby has Rh positive blood (e.g., due to the baby's father having Rh positive blood). During pregnancy, when blood from an Rh positive baby mixes with blood from an Rh negative mother, the mother's body naturally generates antibodies that may affect the current pregnancy with the baby and/or future pregnancies with other babies, in such examples, the antibodies may target and/or attack Rh proteins in the blood of the baby and/or in the blood of any babies of future pregnancies. Such attacks may cause a decrease in red blood cells and potentially cause a plurality of diseases and/or complications (e.g., anemia, jaundice, brain damage, heart failure, or death for the baby(ies).

When an Rh incompatibility exists between a mother and her baby, treatment is available for the mother to prevent generation of antibodies (e.g., by administering injections of Rh immune globulin) and/or destruction of any antibodies that have been generated. Accordingly, the treatment may maintain safety of the baby by protecting the baby's Rh positive blood and or red blood cells. Previous techniques for detecting Rh incompatibility between an Rh negative mother and her Rh positive baby involve various invasive methods (e.g., amniocentesis, including probing the uterus of the mother) and non-invasive methods (e.g., ultrasounds, maternal blood sampling). Invasive methods may be painful and/or, in some instances, dangerous for the mother and/or baby. Previous non-invasive methods, though considered relatively safe, themselves, are performed periodically (e.g., monthly) by a mother's healthcare provider. Accordingly, a considerable amount of time (up to a month or more may pass between an existence of Rh incompatibility taking place and detection of the Rh incompatibility. In some instances, the mothers antibodies may already have caused untreatable damage to the baby and/or the baby's blood system. Thus, it is desirable to detect an Rh incompatibility between a mother and her baby as soon as possible so that the mother may receive treat ensure the safety of the baby.

The bloodstream of the mother during pregnancy may carry cell free deoxyribonucleic acid (DNA) from her baby. As a result of the presence of the cell free DNA, the composition of the mother's blood may change. When an Rh incompatibility exists between a mother (Rh negative) and her baby (Rh positive), the composition of the mothers blood changes due to the presence of an Rh factor in the mothers bloodstream from the cell free DNA. Based on the change in composition of the mother's blood, an amount of light passing through the mother's bloodstream without the presence of an Rh factor would be different than an amount of light passing through the mothers bloodstream with the presence of the Rh factor. Additionally, the presence of the antibodies within the mother's bloodstream may affect the amount of light that passes through the mother's bloodstream. Examples disclosed herein involve monitoring and/or measuring light in a mother's bloodstream to detect an Rh incompatibility between a mother and her baby.

In examples disclosed herein, a wearable device (e.g., a ring, an earring, a wrist band or wrist watch, a fastener, a belt, etc.) may be used to implement an Rh factor incompatibility system. Accordingly, in some examples, the Rh factor incompatibility system may be continuously worn by a pregnant woman throughout her pregnancy (or during a designated period of her pregnancy) to monitor for and/or detect Rh incompatibility between herself and her baby. Furthermore, examples disclosed herein provide for non-invasive, continuous (or near continuous) monitoring of a pregnant mother's bloodstream to detect Rh incompatibility between the mother and her baby. Additionally, examples disclosed herein involve alerting a mother and/or the mothers healthcare provider of an Rh incompatibility between the mother and her baby.

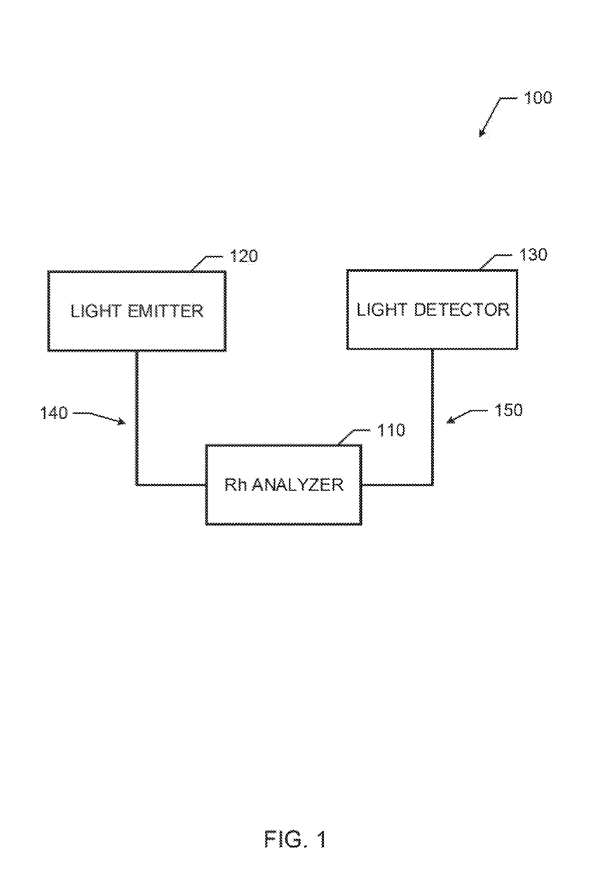

FIG. 1 is a block diagram of an example Rh incompatibility detection system 100 constructed in accordance with the teachings of this disclosure. The Rh incompatibility detection system 100 in the illustrated example of FIG, 1 includes an Rh analyzer 110, a light emitter 120, and a light detector 130. The Rh analyzer 110 communicates with the light emitter 120 and the light detector 130 via the communication links 140, 150. The communication links 140 and/or 150 may be wired and/or wireless communication links. The Rh analyzer 110 uses the light emitter 120 and the light detector 130 to monitor a bloodstream of a mother for Rh incompatibility between the mother and her baby, as disclosed herein. The example Rh analyzer 110 of FIG. 1 instructs the light emitter 120 to emit light into flesh of a mother and the light detector 130 to detect light from the flesh, including the bloodstream, of the mother. The Rh analyzer 110, as disclosed below, uses voltage(s) measured by the light detector 130 to determine whether there is an Rh incompatibility between the mother and her baby (e.g., from a voltage change from one measurement to the next).

The example light emitter 120 of FIG. 1 may be any light emitting device, such as a light emitting diode (LED). The light emitter 120 of FIG. 1 projects light into flesh of a mother, including her bloodstream. Thus, the light emitter 120 causes light to be present in the bloodstream of the mother, which may be detected by the light detector 130. The example light emitter 120 of FIG. 1 may be controlled by the Rh analyzer 110. For example, the light emitter 120 may project light in light bursts periodically (e.g., every minute, every five minutes, every hour, etc.) or a periodically based on instructions from the Rh analyzer 110. In some examples, the light emitter 120 may be a motion activated LED that is powered by human motion, in such examples, the motion of the mother may cause the light emitter 120 to project light or a light burst into the user's flesh. For example, if the Rh incompatibility detection system 100 is worn on a mother's hand, when she moves her hand, a light burst may be projected from the light emitter 120 into the hand for detection by the light detector 130 and analysis by the Rh analyzer 110.

The example light detector 130 of FIG. 1 may be any photodetector, photosensor, light sensor, etc. capable of detecting and/or measuring light and/or electromagnetics in the flesh and/or bloodstream of a mother. The light detector 130 measures a voltage from the detected light in the bloodstream and provides voltage measurements to the Rh analyzer 110 for analysis. In some examples, the light detector 130 may continuously monitor and/or detect light in the mother's bloodstream based on instructions received from the Rh analyzer 110. For example, the light detector 130 may be activated or deactivated depending on when the light emitter 120 is to project light into a mother's flesh. The light detected and/or measured by the light detector 130 may be representative of an amount of cell-free fetal DNA from the baby that is in the bloodstream of the user. In other words, the amount of light measured by the light detector 130 may depend on an amount of cell free DNA from the baby that entered the mothers blood stream.

The Rh analyzer 110 of FIG. 1 monitors a composition of a mother's blood based on light captured in the mother's bloodstream. The light may be caused by the light emitter 120 and detected/measured by the light detector 130. Accordingly, physical locations of the light emitter 120 and the light detector 130 may be configured such that light is projected into and detected within a same portion of the mothers flesh (an ear, a finger, a wrist, a back, a neck, etc.).

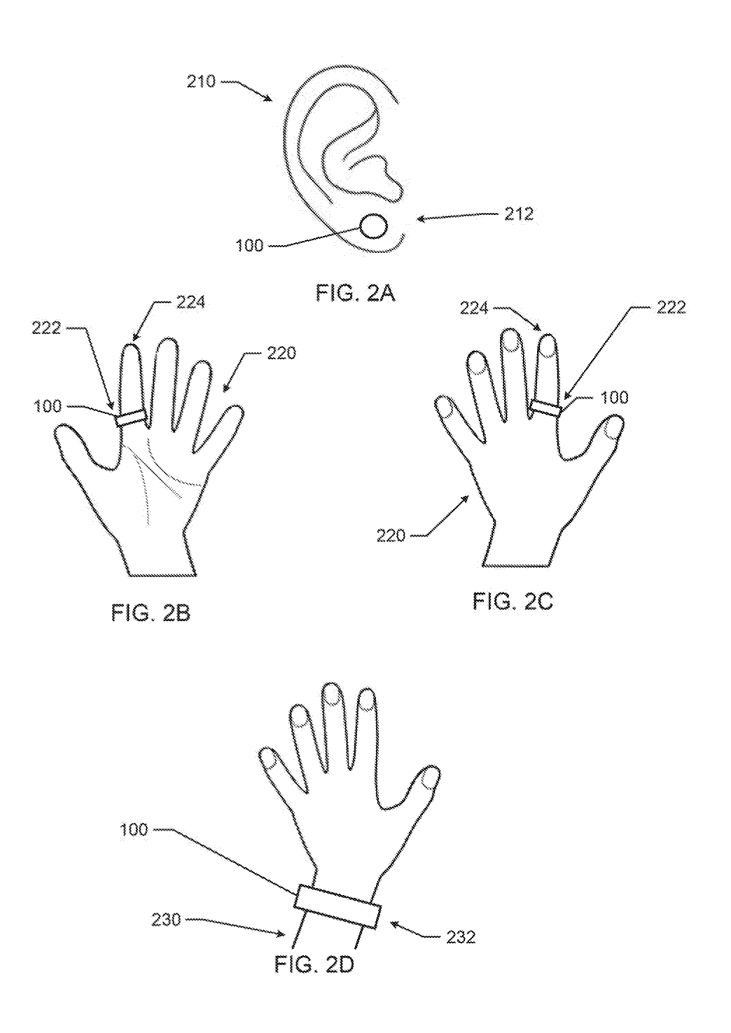

FIGS. 2A-2D illustrate example devices housing the Rh incompatibility detection system 100 of FIG. 1. In FIG. 2A, a front side of an ear 210 is illustrated with an earring 212. In the example of FIG. 2A, the earing 212 may include the light emitter 120 to project light into a first location of the ear 210 (e.g., the illustrated front side) and the light detector 130 to detect or measure light in the ear 210 from a second location of the ear (e.g., the backside). Accordingly, an Rh analyzer 110 of the Rh incompatibility detection system 100 of FIG. 2A may monitor, based on light, a composition of blood in a bloodstream of the ear 210 to detect an Rh incompatibility between the mother and her baby.

FIGS. 2B and 2C illustrate a hand 220 of a mother wearing a ring 222 including the Rh incompatibility detection system 100 of FIG. 1. FIG. 2B illustrates a palm side of the hand 220 and FIG. 2C illustrates a backside of the hand 220. In the example FIGS. 2B and 2C, the ring 222 is on an index finger 224 of the hand 220. The Rh incompatibility detection system 100 of FIGS. 2A and 2B may then include a light emitter 120 on a first location of the index finder 224 (e.g., a location on the backside in FIG. 2C) and a light detector 130 on a second location of the index finger 224 (e.g., a location on the palm side in FIG. 2B). Accordingly, an Rh analyzer 110 of the Rh incompatibility detection system 100 of FIGS. 2B and 2C may monitor, based on light, a composition of blood in a bloodstream of the index finger 224 to detect an Rh incompatibility between the mother and her baby.

Similarly, FIG. 2D illustrates a wrist 230 of a mother wearing a wristband 232 (e.g., a bracelet, a wristwatch, etc.) including the Rh incompatibility system 100 of FIG. 1. In the example of FIG. 20, the wristband 232 may include a light emitter 120 on a first side (e.g., the illustrated top side of the wrist) and a light detector 130 on a second side (e.g., the bottom side of the wrist). Accordingly, an Rh analyzer 110 of the Rh incompatibility detection system 100 of FIG. 20 may monitor, based on light, a composition of blood in a bloodstream of the wrist 230 to detect an Rh incompatibility between the mother and her baby.

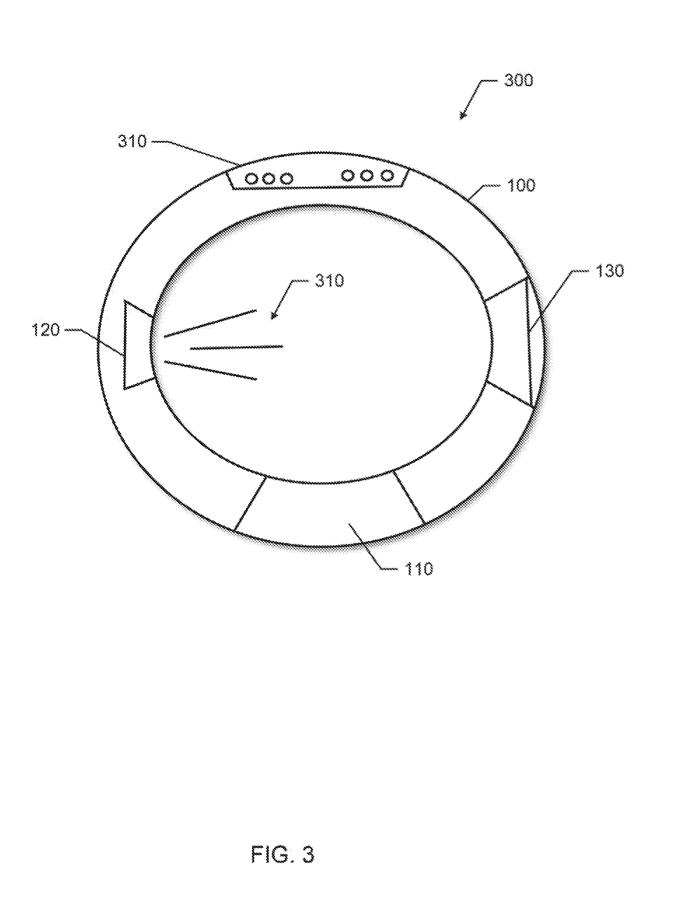

FIG. 3 illustrates an example ring device 300 that may be used to implement the devices of FIGS. 2B-2D. The example ring device 300 of FIG. 3 may be a ring for a user's hand or wristband for user's wrist. The example ring device 300 includes the Rh incompatibility detection system 100 of FIG. 1. In the illustrated example of FIG. 3, the light emitter 120 projects light (denoted by lines 310) inwardly toward the center the ring device 300. Accordingly, when the ring device 300 is worn by a user, the light emitter projects light into flesh of the user's finger, wrist, etc. The light detector 130 may then detect light caused by the projected fight in a bloodstream of the user's finger, wrist, etc. The Rh analyzer 110 of FIG. 3 may then analyze measurements from the light detector 130 to detect Rh incompatibility between the user and her baby.

The example ring device 300 of FIG. 3 also includes an alert device 310. The alert device 310 may be a display (e.g., a liquid crystal display (LCD), an LED display, etc.) and/or a speaker to visually and/or audibly communicate with the user (e.g., to indicate detected Rh incompatibility). In some examples, the alert device may include a communication device to communicate with another device and/or network. For example, the alert device 310 may send Rh incompatibility messages to a user's mobile phone and/or to a device of the user's healthcare provider.

Other example devices in addition to those illustrated in FIGS. 2A-2D and FIG. 3 may be used to implement the Rh incompatibility detection system 100 of FIG. 1. Some additional examples may include, but are not limited to necklaces, headbands, belts, fasteners (e.g., brassiere fasteners, buttons, etc.), gloves, shoes, clothing, etc.

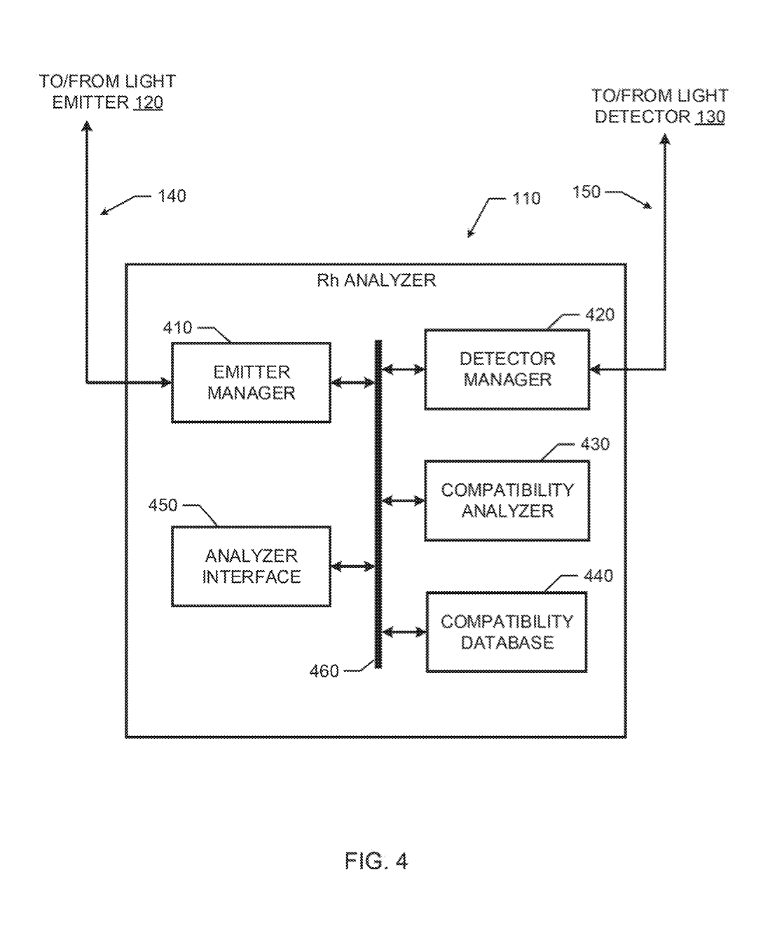

FIG. 4 is a block diagram of an example Rh analyzer 110 that may be used to implement the Rh analyzer 110 of FIG. 1. The Rh analyzer 110 of FIG. 4 includes an emitter manager 410, a detector manager 420, a compatibility analyzer 430, a compatibility database 440, and an analyzer interface 450, in FIG. 4, a communication bus 460 facilitates communication between the emitter manager 410, the detector manager 420, the compatibility analyzer 430, the compatibility database 440, and the analyzer interface 450.

The emitter manager 410 of FIG. 4 facilitates communication with the light emitter 120 of FIG. 1. Accordingly, the emitter manager 410 of FIG. 2 may send instructions to project light based on settings (e.g., settings received from the user via the analyzer interface 150, default settings, etc.) of the Rh analyzer 110. The example detector manager 420 facilitates communication between the Rh analyzer 110 and the light detector 130. The detector manager 420 may instruct the light detector 130 to begin light detection and/or measuring voltage from detected light. Additionally, the detector manager 420 retrieves and/or receives voltages (voltage measurements) from the light detector 130. The detector manager 420 may forward the received voltages to the compatibility analyzer 430.

The example compatibility analyzer 430 of FIG. 4 analyzes measured voltages to determine whether there is an Rh incompatibility between a mother and her baby. The compatibility analyzer 430 determines whether changes in voltage measurements from the light detector 130 correspond to an Rh incompatibility between a mother and her baby. For example, the compatibility analyzer 430 may compare a first voltage received at a first time from the light detector 130 to a second voltage received at a second time (e.g., a time after the first time) from the light detector 130. If the voltages are within a designated range of one another, the compatibility analyzer 430 may determine that there has been no change in the composition of the mother's blood, and thus, no Rh incompatibility exists between the mother and the baby. On the other hand, if the voltages are outside of a designated range (not within the designated range) of one another, the compatibility analyzer 430 may determine that there has been a change in the composition of the mothers blood (due to the presence of an Rh factor in the cell free DNA from the fetus), and thus an Rh incompatibility exists between the mother and the baby.

The compatibility database 440 of FIG. 4 may store incompatibility thresholds for compatibility analysis by the compatibility analyzer 430. Accordingly, the compatibility analyzer 430 may compare voltages or voltage changes to incompatibility thresholds stored in the compatibility database 440 to determine whether measured voltages and/or voltage changes indicate Rh incompatibility or Rh compatibility. For example, an incompatibility threshold may correspond to a designated voltage value, a voltage change, and/or a voltage range. Thus an incompatibility threshold may be satisfied if at least one measured voltage is above, below, or within a designated range set by an incompatibility threshold. Accordingly, if a particular voltage measurement and/or a voltage change a difference between at least two voltage measurements) from the light detector 130 is above or below an incompatibility threshold value, the compatibility analyzer 430 may determine the existence of an Rh incompatibility between the mother and the baby. Additionally or alternatively, if multiple (at least) voltage measurements received from the light detector 130 within a period of time (e.g., a minute, an hour, a day, etc.) are within (or outside) an incompatibility threshold range, the compatibility analyzer 430 may determine that there is an Rh incompatibility between the mother and her baby.

The example incompatibility thresholds in the compatibility database 440 may be set and/or determined based on any appropriate or suitable techniques (e.g., sampling, testing, calibration, etc.). In some examples, incompatibility threshold(s) may be adjusted based on physical characteristics of the mother (and/or baby). For example, a mothers height, weight, etc. may determine values corresponding to certain incompatibility threshold values and/or incompatibility threshold ranges. Additionally or alternatively, stages of the pregnancy (e.g., stages corresponding to trimesters, weeks, months, etc.)and/or size, age, of the baby, etc. may determine the incompatibility thresholds. Accordingly, the incompatibility thresholds may be adjusted for the Rh analyzer 110 automatically and/or manually (e.g., via the analyzer interface 450) by tracking time/length of the pregnancy or other characteristics of the pregnancy (i.e., the incompatibility threshold change as the pregnancy advances toward birth).

Additionally or alternatively, the compatibility database 440 may store other settings for the Rh analyzer 110. For example, the emitter manager 410 may retrieve settings corresponding to a frequency and/or an intensity at which the light emitter 120 is to project light into the flesh of the user. As another example, the detector manager 420 may retrieve settings (e.g., sensitivity, activation, timing, etc.) for detecting light and/or measuring voltage from light within the bloodstream of the user. Such settings may be adjustable based on characteristics of the pregnancy, mother, and/or baby. Additionally, or alternatively, such characteristics may be adjustable based on a housing and/or device implementing the Rh incompatibility detection system 100 of FIG. 1 and/or Rh analyzer 110 of FIG. 4. For example, settings for the light emitter 120, the light detector 130, and/or incompatibility thresholds may be different for a ring (e.g., the ring 222) versus a wrist band (e.g., the wristband 232), perhaps due to the expected distances between the light emitters 120 and the light detectors 130.

The example analyzer interface 450 of FIG. 4 facilitates communication with a user. The analyzer interface 450 may include any type of input device (e.g., a microphone, a button, a keypad, etc.) or output device (e.g., a display, a speaker, etc.) to facilitate control of the Rh, analyzer 110 and/or Rh incompatibility detection system 100. For example, the analyzer interface 450 may include a display (e.g., an LCD, an LED display, etc.) and/or a speaker for visual and/or audible alerts. Such alerts may be used to indicate an Rh incompatibility or Rh compatibility between a mother and her baby.

In some examples, the analyzer interface 450 of FIG. 4 may include a communication module for sending messages to another device (e.g., a smartphone, a computer, a server a network, etc,) of the user and/or the user's healthcare provider. For example, the analyzer interface 450 may communicate with the user's mobile phone to send a message via a cellular network to the users healthcare provider. In some examples, the analyzer interface 450 may similarly stream voltage measurements and/or compatibility analysis from the light detector 130 and/or Rh analyzer 110, respectively to another device and/or network. Accordingly, in such an example, the user and/or the users health care provider may monitor the Rh compatibility or incompatibility of the user.

In some examples, the analyzer interface 450 of FIG. 4 may include and/or communicate with devices for monitoring the pregnancy, the mother, and/or the baby (e.g., heart rate monitors, blood pressure devices, etc.). Accordingly, information/data from these devices may be used to adjust settings (e.g., incompatibility thresholds) of the Rh analyzer 110 and/or communicate with the mother via the analyzer interface 450.

While an example manner of implementing the Rh analyzer 110 of FIG. 1 is illustrated in FIG. 4, at least one of the elements, processes and/or devices illustrated in FIG. 1 may be combined, divided, re-arranged, omitted, eliminated and/or implemented in any other way. Further, the emitter manager 410, the detector manager 420, the compatibility analyzer 430, the compatibility database 440, the analyzer interface 450 and/or, more generally, the example Rh analyzer 110 of FIG. 4 may be implemented by hardware, software, firmware and/or any combination of hardware, software and/or firmware. Thus, for example, any of the emitter manager 410, the detector manager 420, the compatibility analyzer 430, the compatibility database 440, the analyzer interface 450 and/or, more generally, the example Rh analyzer 110 could be implemented by at least one of an analog or digital circuit, a logic circuit, a programmable processor, an application specific integrated circuit (ASIC), a programmable logic device (PLD) and/or a field programmable logic device (FPLD). When reading any of the apparatus or system claims of this patent to cover a purely software and/or firmware implementation, at least one of the emitter manager 410, the detector manager 420 the compatibility analyzer 430, the compatibility database 440, the analyzer interface 450 is/are hereby expressly defined to include a tangible computer readable storage device or storage disk such as a memory, a digital versatile disk (DVD), a compact disk (CD), a Blu-ray disk, storing the software and/or firmware. Further still, the example Rh analyzer 110 of FIG. 4 may include at least one element, process, and/or device in addition to, or instead of, those illustrated in FIG. 4, and/or may include more than one of any or all of the illustrated elements, processes and devices.

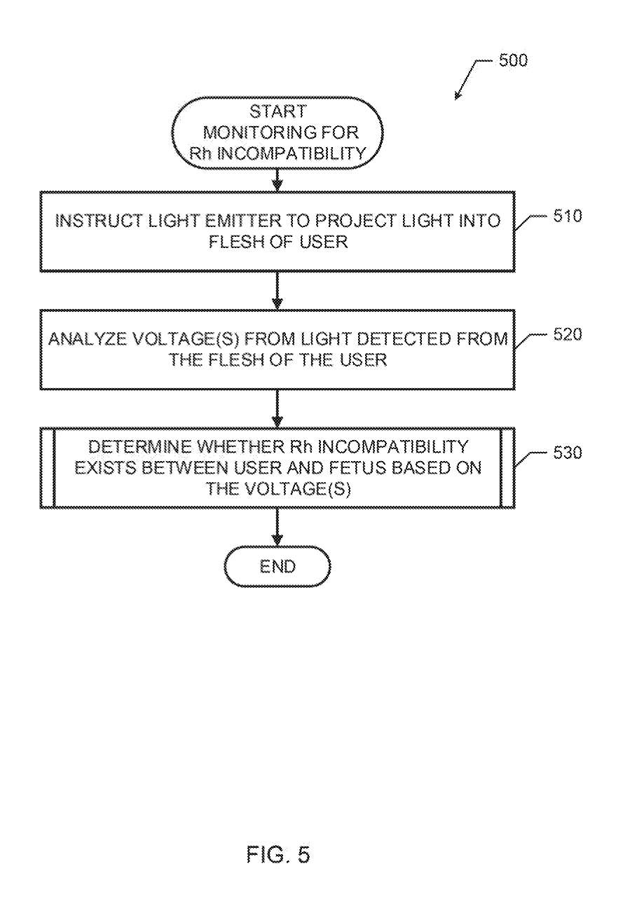

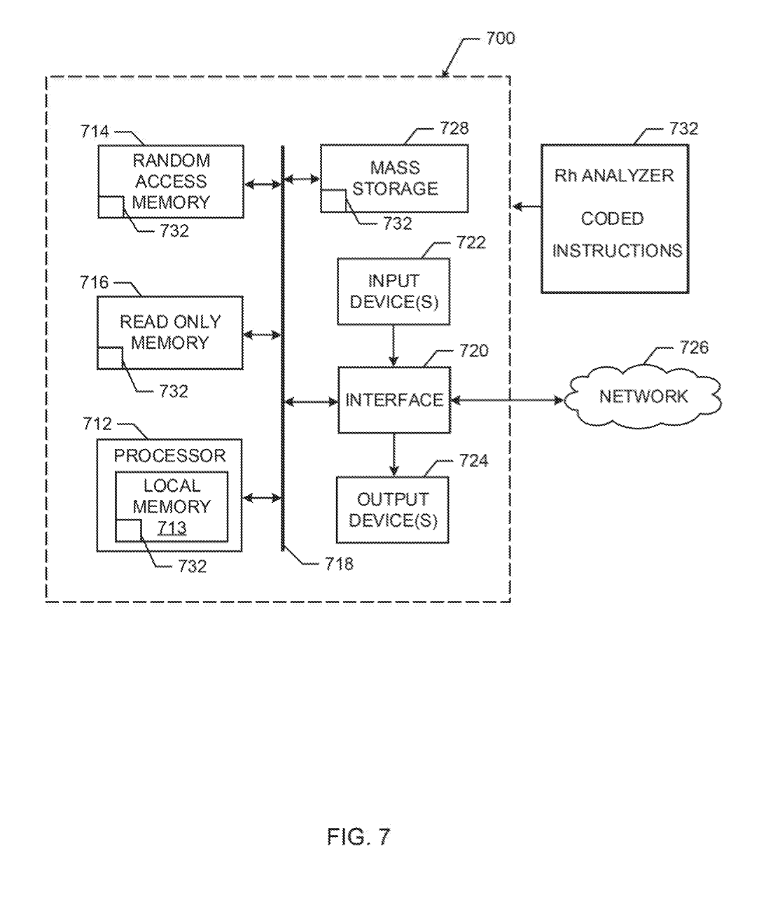

Flowcharts representative of example machine readable instructions for implementing the Rh analyzer of FIGS. 1 and/or 4 are shown in FIGS. 5 and/or 6. In this example, the machine readable instructions comprise a program/process for execution by a processor such as the processor 712 shown in the example processor platform 700 discussed below in connection with FIG. 7. The program/process may be embodied in software stored on a tangible computer readable storage medium such as a CD-ROM, a floppy disk, a hard drive, a digital versatile disk (DVD), a Blu-ray disk, or a memory associated with the processor 712, but the entire program/process and/or parts thereof could alternatively be executed by a device other than the processor 712 and/or embodied in firmware or dedicated hardware. Further, although the example program is described with reference to the flowchart illustrated in FIGS. 5 and/or 6, many other methods of implementing the example Rh analyzer 110 may alternatively be used. For example, the order of execution of the blocks may be changed, and/or some of the blocks described may be changed, eliminated, or combined.

The example process 500 of FIG. F begins with an initiation of the Rh analyzer 110 (e.g., upon powering up the Rh incompatibility detection system 100, upon activation from a user, upon instructions from a user, upon motion from a user, etc.). At block 510 of FIG. 5, the emitter manager 410 instructs the light emitter to project light into flesh of a user. For example, at block 510, the emitter manager 410 may send the instructions based on settings of the Rh analyzer 110 to check for Rh incompatibility periodically (e.g., every 10 minutes, every 30 minutes, every hour, etc.) or a periodically (e.g., upon motion detection, upon instructions from a user, upon instructions from a device in communication with Rh analyzer 110, etc.).

In FIG. 5, at block 520, the example compatibility analyzer 430 analyzes voltage(s) from light detected by the light detector 130 from the flesh of the user. For example, at block 520, the compatibility analyzer 430 may compare received voltages to each other to identify a voltage change (i.e., to determine a difference between the voltages), and/or to incompatibility thresholds stored in the compatibility database 440. At block 530, the compatibility analyzer 430 determines whether an Rh incompatibility exists between the user and a fetus in a womb of the user (i.e., between mother and baby) based on the voltages. After block 530, the process 500 of FIG. 5 ends. In some examples, after block 530 of FIG. 5, the Rh analyzer 110, via the analyzer interface 450, may alert a user and/or a healthcare provider of the user that an Rh incompatibility exists between a mother and her baby. In some examples, the process 500 of FIG. 5 may be iteratively executed and continue analyzing and/or monitoring for Rh incompatibility between a mother and her baby.

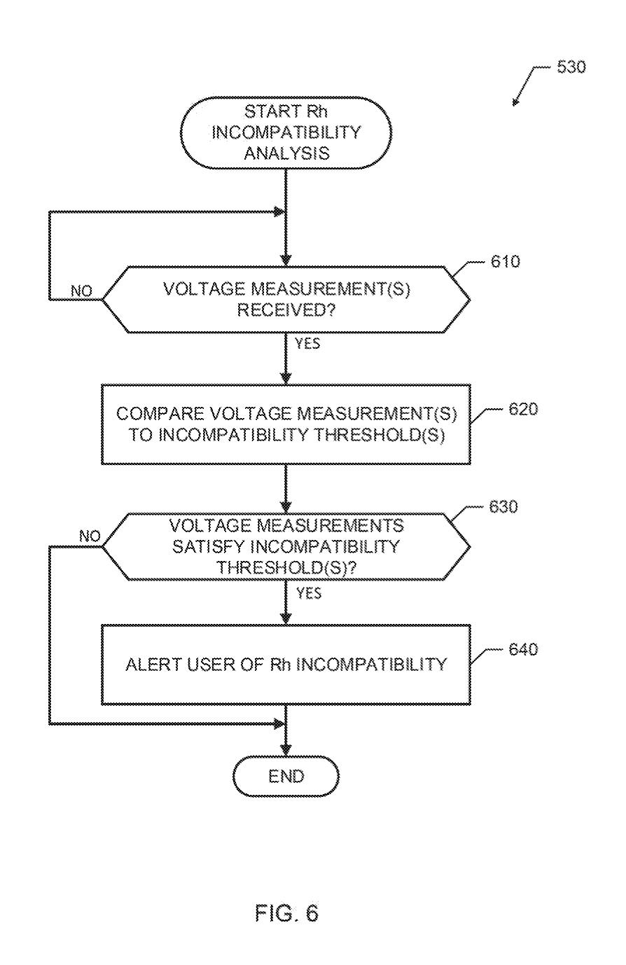

FIG. 6 illustrates an example process 530 that may be executed to implement block 520 and/or block 530 of FIG. 5. The process 530 begins with an initiation of the Rh analyzer 110. At block 610, the compatibility analyzer 430 determines whether voltage measurement(s) have been received from the light detector 130 and/or the detector manager 420. In some examples, at block 610, a single voltage measurement may be received or a plurality of voltage measurements may be received. If no voltage measurements have been received, control returns to block 610, and the compatibility analyzer 430 continues to monitor for received voltage measurements. If voltage measurement(s) have been received at block 610, the compatibility analyzer 430 compares the voltage measurement(s) to incompatibility threshold(s) (block 620). For example, at block 620, the compatibility analyzer 430 may compare a voltage measurement to an incompatibility threshold to determine whether the voltage measurement is above, below, or within range of an incompatibility threshold. In some examples, at block 620, the compatibility analyzer 430 may determine whether a plurality of voltage measurements (e.g., a first measurement received first time and a second measurement received at a second later time) are in an incompatibility threshold range.

At block 630 in the example of FIG. 6, the compatibility analyzer 430 determines whether the voltage measurement(s) satisfy the incompatibility threshold(s) (i.e., determines whether the measurement(s) are above an incompatibility threshold, below an incompatibility threshold, within an incompatibility threshold range or outside of an incompatibility threshold range). If, at block 630, the voltage measurement(s) do not satisfy the incompatibility threshold(s), the process 530 of FIG. 6 ends. If the voltage measurement(s) do satisfy the incompatibility thresholds at block 630, the compatibility analyzer 430 and/or the analyzer interface 450 alert the user of an Rh incompatibility (block 640). After block 640, the example process 530 of FIG. 6 ends. In some examples, the process 530 may be iteratively executed to continue analyzing and/or monitoring for Rh incompatibility between a mother and her baby.

As mentioned above, the example processes of FIGS. 5 and/or 6 may be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example processes of FIGS. 5 and/or 6 may be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching, of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended.

FIG. 7 is a block diagram of an example processor platform 700 capable of executing the instructions of FIGS. 5 and/or 6 to implement the Rh analyzer 110 of FIG. 4. The processor platform 700 may be located on any device (e.g., the earring 212 of FIG. 2A, the ring 222 of FIGS. 2B and 2C, the wristband 232 of FIG. 2D, and/or the rind device 300 of FIG. 3) for implementing the Rh incompatibility detection system 100 of FIG. 1. The processor platform 700 can be, for example, a mobile device (e.g., a smart watch), a smart wearable device, or any other type of computing device.

The processor platform 700 of the illustrated example of FIG. 7 includes a processor 712. The processor 712 of the illustrated example is hardware. For example, the processor 712 can be implemented by at least one integrated circuit, logic circuit, microprocessor or controller from any desired family or manufacturer.

The processor 712 of the illustrated example includes a local memory 713 (e.g., a cache). The processor 712 of the illustrated example is in communication with a main memory including a volatile memory 714 and a non-volatile memory 716 via a bus 718. The volatile memory 714 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 716 may be implemented by flash memory and/or any outer desired type of memory device. Access to the main memory 714, 716 is controlled by a memory controller. The main memory 714, 716 may implement the compatibility database 440 of FIG. 4.

The processor platform 700 of the illustrated example also includes an interface circuit 720. The interface circuit 720 may be implemented by any type of interface standard, such as an Ethernet interface, a universal serial bus (USB), and/or a PCI express interface.

In the illustrated example, at least one input device 722 is connected to the interface circuit 720. The input device(s) 722 permit(s) a user to enter data and commands into the processor 712. The input device(s) can be implemented by, for example, an audio sensor, a microphone, a camera (still or video), a keyboard/keypad, a button, a touchscreen, a track-pad, a trackball, isopoint and/or a voice recognition system.

At least one output device 724 is also connected to the interface circuit 720 of the illustrated, example. The output device(s) 724 can be implemented, for example, by display devices (e.g., a light emitting diode (LED), an organic light emitting diode (OLED), a liquid crystal display, a touchscreen, a tactile output device, a light emitting diode (LED), and/or speakers). The interface circuit 720 of the illustrated example, thus, may include a graphics driver card, a graphics driver chip or a graphics driver processor.

The interface circuit 720 of the illustrated example also includes a coma communication device such as a transmitter, a receiver, a transceiver, a modem and/or network interface card to facilitate exchange of data with external machines (e.g., computing devices of any kind) via a network 726 (e.g., an Ethernet connection, a digital subscriber line (DSL), a telephone line, coaxial cable, a cellular telephone system, etc.).

At least one of the interface circuit 720, the input device(s) 722, or the output device(s) 724 may implement the analyzer interface 450 of FIG. 4.

The processor platform 700 of the illustrated example also includes at least one mass storage device 728 for storing software and/or data. Examples of such mass storage device(s) 728 include floppy disk drives, hard drive disks, compact disk drives, Blu-ray disk drives, RAID systems, and digital versatile disk (DVD) drives.

The coded instructions 732 of FIGS. 4 and/or may be stored in the mass storage device 728, in the local memory 713 in the volatile memory 714, in the nonvolatile memory 716, and/or on a removable tangible computer readable storage medium such as a CD or DVD.

From the foregoing, it will be appreciated that the above disclosed methods, apparatus and articles of manufacture provide an Rh incompatibility detection system. Examples disclosed herein enable a pregnant mother to continuously or substantially continuously monitor whether her blood is Rh compatible or Rh incompatible with her baby's blood. Furthermore, the examples disclosed herein non-invasively project light into flesh (including a bloodstream) and analyze light from the flesh (including the bloodstream) caused by the projected to light to detect Rh incompatibility between a mother and a baby. Examples disclosed herein may be implemented via wearable devices that may not intrude on a mother's pregnancy and normal activity.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. A system comprising:
a light emitter to project light into flesh of a user;
a light detector to measure voltage from tight in the flesh of the user caused by the projected light; and
an Rh analyzer to detect Rh incompatibility between the user and a fetus within the user based on the voltage,

2. The system of claim 1, wherein the voltage is a first voltage and the light detector is to measure a second voltage from the light in the flesh of the user, the first voltage measured at a first time and the second voltage measured at a second time different from the first time, the Rh analyzer to detect the incompatibility when the first voltage and the second voltage satisfy an incompatibility threshold.

3. The system of claim 2, wherein the first voltage and second voltage satisfy an incompatibility threshold when the first voltage and the second voltage are outside of a designated voltage range.

4. The system of claim 1, wherein the voltage measured from the light in the flesh is representative of an amount of cell-free fetal DNA from the fetus that is within a bloodstream of the user.

5. The system of claim wherein
the light emitter is further to repeatedly project the light as light bursts into the flesh of the user,
the light detector is further to repeatedly measure voltages corresponding to the light bursts, and
the Rh analyzer is further to detect the Rh incompatibility based on a comparison of the repeatedly measured voltages.

6. The system of claim 1, wherein the detected Rh incompatibility indicates that an Rh factor of the user is Rh-negative and that an Rh factor of the baby is Rh-positive.

7. A method comprising:
instructing a light emitter to emit light into flesh of a user;
analyzing voltages from light detected from the flesh of the user, the light detected from the flesh of the user caused by the light emitted into the flesh of the user; and
detecting whether an Rh incompatibility exists between the user and a fetus in a womb of the user based on the measured voltages.

8. The method of claim 7, further comprising:
detecting a voltage change between at least two voltage measurements of the voltages; and
determining that the Rh incompatibility exists based on the voltage change.

9. The method of claim wherein a value of the voltage change satisfies an incompatibility threshold.

10. The method of claim 7, further comprising: determining whether the Rh factor of the user is incompatible with the Rh factor of the fetus based on physic characteristics of the user.

11. The method of claim 10, further comprising:
receiving physical characteristics of the user; and
monitoring for a voltage change between two measurements of the measured voltages based on the physical characteristics of the user, the voltage change representative of the Rh incompatibility.

12. The method of claim 7, further comprising alerting the user when the Rh incompatibility exists.

13. A non-transitory computer readable storage medium comprising instructions that, when executed, cause a machine to at least:
instruct a light emitter to emit light into flesh of a user at a first frequency;
analyze voltages from light detected from the flesh of, the user at the first frequency, the light detected from the flesh of the user caused by the light emitted into the flesh of the user; and
detect that an Rh factor of the user is incompatible with an Rh factor of a fete in a womb of the user based on the measured voltages.

14. The non-transitory computer readable storage medium of claim 13, wherein the instructions, when executed, cause the machine to:
compare a first and second voltage measurement from the measured voltages to determine a voltage change between the first and second voltage measurements; and
determine that the Rh factor of the user is incompatible with the Rh factor of the fetus when the voltage change satisfies an incompatibility threshold.

15. The non-transitory computer readable storage medium of claim 14, wherein the instructions, when executed, cause the machine to alert the user when the Rh factor of the user is incompatible with the Rh factor of the fetus.
